(19) United States
(12) Patent Application Publication
(10) Pub. No.: US 2022/0237940 A1
(43) Pub. Date: Jul. 28, 2022

(54) ULTRASONIC IMAGING DEVICE AND METHOD FOR IMAGE ACQUISITION IN THE ULTRASONIC DEVICE

(71) Applicant: FINGERPRINT CARDS ANACATUM IP AB, GÖTEBORG (SE)

(72) Inventors: Hamed BOUZARI, KØBENHAVN Ø (DK); Farzan GHAVANINI, MÖLNDAL (SE)

(73) Assignee: FINGERPRINT CARDS ANACATUM IP AB, GÖTEBORG (SE)

(21) Appl. No.: 17/615,137

(22) PCT Filed: Jun. 1, 2020
(86) PCT No.: PCT/SE2020/050552

§ 371 Date: Nov. 30, 2021

(30) Foreign Application Priority Data
Jun. 10, 2019 (SE) 1950682-3

(51) Int. Cl. G06V 40/13; G06F 3/043

(52) U.S. Cl. CPC G06V 40/1306; A61B 5/1172; G06F 3/0436

(57) ABSTRACT

Method for image acquisition in an ultrasonic biometric imaging device, the device comprising a plurality of ultrasonic transducers arranged at a periphery of a touch surface along one side of the touch surface, the method comprising: determining a target area of a touch surface; identifying a blocking feature preventing ultrasonic wave propagation in the touch surface such that the blocking feature creates a blocked region in the touch surface where image acquisition is not possible; determining that the target area at least partially overlaps the blocked region; dividing the transducers into a first subset and a second subset, the first and second subset being defined in that ultrasonic waves emitted by the respective subset reaches the target area on a first and second side of the blocking feature; and capturing an image of the biometric object using transmit and receive beamforming.

### FIELD OF THE INVENTION

The present invention relates to an ultrasonic imaging device and to a method for image acquisition in an ultrasonic device. In particular, the present invention relates to forming an image based on ultrasonic reflections in the imaging device.

### BACKGROUND OF THE INVENTION

Biometric systems are widely used as means for increasing the convenience and security of personal electronic devices, such as mobile phones etc. Fingerprint sensing systems in particular are now included in a large proportion of all newly released personal communication devices, such as mobile phones.

Due to their excellent performance and relatively low cost, capacitive fingerprint sensors have been used in an overwhelming majority of all biometric systems.

Among other fingerprint sensing technologies, ultrasonic sensing also has the potential to provide advantageous performance, such as the ability to acquire fingerprint (or palmprint) images from very moist fingers etc.

One class of ultrasonic fingerprint systems of particular interest are systems in which acoustic signals are transmitted along a surface of a device element to be touched by a user, and a fingerprint (palm print) representation is determined based on received acoustic signals resulting from the interaction between the transmitted acoustic signals and an interface between the device member and the user's skin.

Such ultrasonic fingerprint sensing systems, which are, for example, generally described in US 2017/0053151 may provide for controllable resolution, and allow for a larger sensing area, which may be optically transparent, without the cost of the fingerprint sensing system necessarily scaling with the sensing area and thereby allowing integration of ultrasonic fingerprint sensors in a display of a device.

However, current solutions struggle to provide a high-resolution fingerprint with a large coverage area of the full in-display screen, as it is difficult to handle and process the large amount of RF-data generated for each touch event and thereby apply the image reconstruction and matching procedures required.

Accordingly, there is a need for improved methods and systems for large area fingerprint imaging using ultrasonic technology.

### SUMMARY

In view of above-mentioned and other drawbacks of the prior art, it is an object of the present invention to provide a method and system for image acquisition in an ultrasonic biometric imaging device which is capable of adapting the imaging acquisition process based on properties of a touch surface.

According to one embodiment of the invention, there is provided a method for image acquisition in an ultrasonic biometric imaging device. The device comprises a plurality of ultrasonic transducers arranged at a periphery of a touch surface along one side of the touch surface. The method comprises: determining a target area of a touch surface; identifying a blocking feature preventing ultrasonic wave propagation in or at the touch surface such that the blocking feature creates a blocked region in the touch surface where image acquisition is not possible; determining that the target area at least partially overlaps the blocked region; dividing the plurality of transducers into a first subset and a second subset, the first subset being defined in that ultrasonic waves emitted by the first subset reaches the target are on a first side of the blocking feature and the second subset being defined in that ultrasonic waves emitted by the second subset reaches the target area on a second side of the blocking feature; controlling the first and second subset of transducers to emit a first and a second ultrasonic beam towards the target area using transmit beamforming, the ultrasonic beam being a defocused or unfocused ultrasonic beam; by the ultrasonic transducers, receiving reflected ultrasonic echo signals defined by received RF-data, the reflected ultrasonic echo signals resulting from interactions with an object in contact with the touch surface at the target area; subtracting background RF-data from the received RF-data to form a clean image; performing receive side beamforming to form a reconstructed image from the clean image; and for a plurality of reconstructed images resulting from a plurality of emitted ultrasonic beams for a given target area, adding the plurality of reconstructed images to form a summed image.

The present method is aimed at acquiring an image of a biometric object such as a fingerprint or palm print when a finger or a palm is placed in contact with the touch surface. The touch surface may for example be a surface of a display cover glass in a smartphone, tablet or the like. However, the described method can equally well be implemented in other devices, such as an interactive TV, meeting-table, smart-board, information terminal or any other device having a transparent or non-transparent cover structure where ultrasonic waves can propagate. Since the transducers are arranged at the periphery of the active touch surface, the described method can also be employed in e.g. an interactive shop window or a display cabinet in a store, museum or the like. The biometric object may in some applications be the cheek or ear of a user.

Transmit beamforming may mean using a number of transducer elements in a transmit step so that by adjusting transmission delays of the respective transducers, a defocused or unfocused ultrasonic beam is generated and emitted towards the target area. The directionality of the resulting ultrasonic beam is limited by the opening angle of the respective transducers used to form the beam.

The ultrasonic transducers typically comprise a piezoelectric material generating an ultrasonic signal in response to an electric field applied across the material by means of the top and bottom electrodes. In principle, it is also possible to use other types of ultrasonic transducers, such as capacitive micromachined ultrasonic transducers (CMUT) or piezoelectric micromachined ultrasonic transducers (PMUT). The ultrasonic transducers will be described herein as transceivers being capable of both transmitting and receiving ultrasonic signals. However, it is also possible to form a system comprising individual and separate ultrasonic transmitters and receivers.

The device is further considered to comprise ultrasonic transducer control circuitry configured to control the transmission and reception of ultrasonic signals and considered to comprise appropriate signal processing circuitry required for extracting an image from the received ultrasonic echo signals.

The ultrasonic signals can be described by radio frequency data, RF-data. The radio spectrum may encompass frequencies from 3 Hz up to 3 THz, and for ultrasonic signals the applicable frequency range is approximately 20 kHz up to several GHz, such as 3 GHz. Accordingly, the received RF-data describes an oscillating signal resulting from the echo of the emitted ultrasonic beam. Similarly, background RF-data describes the received ultrasonic signal for an emitted ultrasonic beam for the case when there is no object in contact with the touch surface. Which ultrasonic frequency or frequency rage to use is determined based on the application at hand and may vary depending on parameters such as required resolution, type of transducer, material in which the ultrasonic signal will propagate, power consumption requirements etc.

The present invention is based on the realization that by using a method for image acquisition including both transmit and receive beamforming it is possible to control the emitted ultrasonic signal to make it possible to capture an image of an object in contact with the touch surface at an area which would otherwise be obscured by a blocking feature of the touch surface. In other words, the diffractive properties of sound waves propagating in a solid material are utilized to in essence see around corners. By using beamforming in combination with a defocused or unfocused beam, it is thus possible to capture an image of an object located in a “hidden region” where plane-wave propagation is prevented by a blocking feature of the touch surface. An unfocused beam is a beam which is controlled by beamforming to neither diverge nor converge while propagating towards the target area. There will however be some divergence due to diffraction. A defocused beam is a diverging beam which is controlled by beamforming to appear to originate from a virtual point source located behind the ultrasonic transducers.

According to one embodiment of the invention, forming a defocused beam comprises performing transmit beamforming to form a virtual point source located behind the transducers and outside of the touch surface. Thereby, the emitted beam will have a cone shape where the tip of the cone is located at the virtual point source, meaning that the beam shape when seen in the touch surface will have the shape of a truncated cone.

According to one embodiment of the invention, the method may further comprise emitting a respective first and second directional defocused beam by the first and second subset of transducers such that the blocked region is minimized. The emitted beams are thereby shaped based on the known properties of the blocking feature such that the blocking region is minimized or even eliminated. Based on knowledge of the blocking feature, a blocking region for non-directional emitted ultrasonic beams can be estimated, and the beams can be suitably adjusted to minimize said blocking region.

According to one embodiment of the invention, the method may further comprise emitting a respective first and second directional defocused beam by the first and second subset of transducers, wherein the first and second directional defocused beams have the same shape. For a symmetrical blocking feature as seen from the plurality of transducers, such as a circular opening, it is preferable to use two beams having the same shape reaching the target area from opposite sides of the blocking feature.

According to one embodiment of the invention, the method may further comprise controlling the ultrasonic transducers to emit a defocused beam or an unfocused beam based on a speed of sound in the touch surface. The diffraction properties of the emitted ultrasonic waves are dependent on the propagation velocity in the material in which the waves propagate, and the relation between wavelength λ, propagation speed v_{US} and frequency f is described as λ=v_{US}/f. Since the wavelength should be the same to achieve the desired resolution, a lower propagation speed means that the frequency can be proportionally lowered while maintaining the same resolution. An unfocused beam would exhibit more dispersion at a lower frequency compared to at a higher frequency, thereby making it more feasible to use an unfocused beam at lower frequencies. An example wavelength for fingerprint recognition may be approximately 175 μm, which for a propagation speed of 1750 m/s gives a frequency of 10 MHz. The acoustic energy for an unfocused beam drops with a ratio proportional to 1/√r, where r, is the distance from the transducer to the wavefront. For a defocused beam, the energy drop is proportional to 1/r, which means a faster loss of energy with distance. Accordingly, it is more preferable to use an unfocused beam if possible.

According to one embodiment of the invention, the touch surface may be a surface of a display panel and the blocking feature is an opening in the display panel. The blocking feature may for example be a cutout in the display panel for a speaker or microphone, or it may a cutout or an opening for a camera. For such blocking features, it can be assumed that the properties of the blocking feature is known to the biometric imaging system, and that the properties of the blocking region are equally known, so that the imaging system can accommodate for the blocking region without having to make any measurement or calibration.

Accordingly, identifying a blocking feature may comprise retrieving stored information describing properties of the blocking feature, such as if the blocking feature is an integral part of the device in which the biometric imaging device is arranged.

According to one embodiment of the invention, identifying a blocking feature may comprise forming an image of at least a portion of the touch surface, detecting a blocking feature in the formed image and determining properties of the blocking feature based on the formed image. Thereby, properties of the blocking feature and blocking region can be determined even if the imaging system has no prior knowledge of a blocking feature. This is for example advantageous in situations where a blocking feature is suddenly formed in the touch surface. Such a blocking feature may be a scratch or a crack in a display glass. A feature which is detected in an image can be defined as a blocking feature if it is sufficiently prominent and if it negatively impacts detection of the biometric object.

According to one embodiment of the invention, emitting a first and a second ultrasonic beam towards the target area using transmit beamforming may comprise emitting a first and a second ultrasonic beam having the largest possible angles in relation to the blocking feature, which will act to minimize the blocking region.

According to one embodiment of the invention, determining the target area comprises receiving information describing the target area from a touch sensing arrangement configured to detect a location of an object in contact with the touch surface. Thereby, the biometric imaging device does not have to be used to detect a target area, having the effect that the biometric imaging device may be in an idle mode or sleep mode until a target area is detected. The touch sensing arrangement may also be used to determine properties of a blocking feature such that a blocking region can be determined based on input from the touch sensing arrangement.

According to a second aspect of the invention, there is provided an ultrasonic biometric imaging device comprising: a cover structure comprising a touch surface; a plurality of ultrasonic transducers arranged at a periphery of the touch surface, the plurality of ultrasonic transducers being configured to emit a defocused or unfocused ultrasonic beam towards a target area using transmit beamforming and to receive a reflected ultrasonic echo signals defining received RF-data, the reflected ultrasonic echo signals resulting from reflections by an object in contact with the touch surface at the target area; and a biometric imaging control unit.

The biometric imaging control unit is configured to: determine a target area of a touch surface; identify a blocking feature preventing ultrasonic wave propagation in or at the touch surface such that the blocking feature creates a blocked region in the touch surface where image acquisition is not possible; determine that the target area at least partially overlaps the blocked region; divide the plurality of transducers into a first subset and a second subset, the first subset being defined in that ultrasonic waves emitted by the first subset reaches the target are on a first side of the blocking feature and the second subset being defined in that ultrasonic waves emitted by the second subset reaches the target area on a second side of the blocking feature; control the first and second subset of transducers to emit a first and a second ultrasonic beam towards the target area using transmit beamforming, the ultrasonic beam being a defocused or unfocused ultrasonic beam; by the ultrasonic transducers, receive reflected ultrasonic echo signals defined by received RF-data, the reflected ultrasonic echo signals resulting from interactions with an object in contact with the touch surface at the target area; subtract background RF-data from the received RF-data to form a clean image; perform receive side beamforming to form a reconstructed image from the clean image; and for a plurality of reconstructed images resulting from a plurality of emitted ultrasonic beams for a given target area, add the plurality of reconstructed images to form a summed image.

According to one embodiment of the invention the blocking feature preventing ultrasonic wave propagation is a cutout in the cover structure located at the edge of the cover structure, and wherein the first subset of ultrasonic transducers is located at a first side of the cutout and the second subset of ultrasonic transducers is located at a second side of the cutout, opposite the first side. By arranging the transducer at respective sides of the blocking features it is possible to direct the emitted ultrasonic beams from sides of the blocking feature to best minimize the blocked region.

The blocking feature preventing ultrasonic wave propagation may for example be an opening in the cover structure located at the edge of the cover structure or a crack in the cover structure located at the edge of the cover structure, and the cover structure may be a display glass in a user device such as a smartphone.

According to one embodiment of the invention, the plurality of transducers may be arranged in a single row on a single side of the touch surface. By means of the described method and system utilizing transmit and receive beamforming, it can possible to acquire an image from the entire touch area using transducers on only one side of the touch surface. Naturally, this also depends on other factors such as size of the touch surface and power of the transducers.

Additional effects and features of the second aspect of the invention are largely analogous to those described above in connection with the first aspect of the invention.

Further features of, and advantages with, the present invention will become apparent when studying the appended claims and the following description. The skilled person realize that different features of the present invention may be combined to create embodiments other than those described in the following, without departing from the scope of the present invention.

### BRIEF DESCRIPTION OF THE DRAWINGS

These and other aspects of the present invention will now be described in more detail, with reference to the appended drawings showing an example embodiment of the invention, wherein:

FIG. 1A schematically illustrates a display arrangement comprising a biometric imaging device according to an embodiment of the invention;

FIG. 1B is a cross section view of a display arrangement comprising a biometric imaging device according to an embodiment of the invention;

FIG. 2 is a flow chart outlining the general steps of a method for acquiring an image according to an embodiment of the invention;

FIGS. 3A-B schematically illustrate a biometric imaging device according to embodiments of the invention; and

FIGS. 4A-C schematically illustrate features of a biometric imaging device according to an embodiment of the invention.

### DETAILED DESCRIPTION OF EXAMPLE EMBODIMENTS

In the present detailed description, various embodiments of the system and method according to the present invention are mainly described with reference to a biometric imaging device adapted to form an image of a finger placed on a display glass of a smartphone. It should however be noted that the described technology may be implemented in a range of different applications.

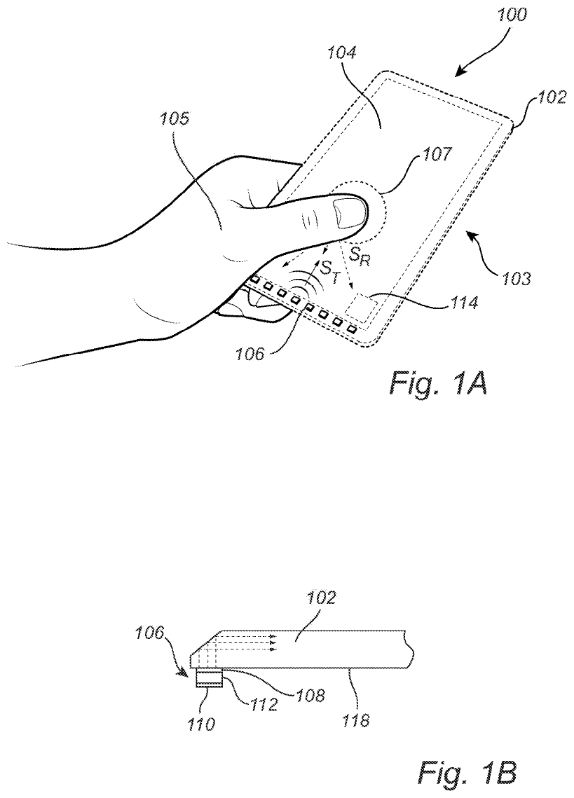

FIG. 1A schematically illustrates a biometric imaging device 100 integrated in an electronic device in the form of a smartphone 103. The illustrated smartphone 103 comprises a display panel having a cover structure 102 in the form of a cover glass 102. The cover glass 102 defines an exterior surface 104 configured to be touched by a finger 105, herein referred to as the touch surface 104. The cover structure 102 is here illustrated as a transparent cover glass 102 of a type commonly used in a display panel of the smartphone 103. However, the cover structure 102 may equally well be a non-transparent cover plate as long as the acoustic properties of the cover structure 102 allows for propagation of ultrasound energy.

The display arrangement further comprises a plurality of ultrasonic transducers 106 connected to the cover structure 102 and located at the periphery of the cover structure 102. Accordingly, the ultrasonic transducers 106 are here illustrated as being non-overlapping with an active sensing area of the biometric imaging device formed by the ultrasonic transducers 106 and the cover structure 103. However, the ultrasonic transducers 106 may also be arranged and configured such that they overlap an active sensing area. FIG. 1A illustrates an example distribution of the transducers 106 where the transducers 106 are evenly distributed along one edge of the cover structure 102. However, other transducer distributions are equally possible, such as arranging the transducers 106 on two, three or four sides of the display panel, and also irregular distributions are possible.

The distribution of transducers may for example be selected based on the size of the desired area. For a typical display in a smartphone or the like, it may for example be sufficient to arrange transducers along the top and bottom edges of the display to achieve full area coverage.

FIG. 1B is a cross section view of the cover structure 102 where it is illustrated that the ultrasonic transducers 106 are arranged underneath the cover structure 102 and attached to the bottom surface 118 of the cover structure 102. The ultrasonic transducer 106 is a piezoelectric transducer comprising a first electrode 108 and second electrode 110 arranged on opposing sides of a piezoelectric element 112 such that by controlling the voltage of the two electrodes 108, 110, an ultrasonic signal can be generated which propagates into the cover structure 102.

The pitch of the transducers may be between half the wavelength of the emitted signal and 1.5 times the wavelength, where the wavelength of the transducer is related to the size of the transducer. For an application where it is known that beam steering will be required, the pitch may preferably be half the wavelength so that grating lobes are located outside of an active imaging area. A pitch approximately equal to the wavelength of the emitted signal may be well suited for applications where no beam steering is required since the grating lobes will be close to the main lobe. The wavelength of the transducer should be approximately equal to the size of the features that are to be detected, which in the case of fingerprint imaging means using a wavelength in the range of 50-300 μm. An ultrasonic transducer 106 can have different configurations depending on the type of transducer and also depending on the specific transducer package used. Accordingly, the size and shape of the transducer as well as electrode configurations may vary. It is furthermore possible to use other types of devices for the generation of ultrasonic signals such as micromachined ultrasonic transducers (MUTs), including both capacitive (cMUTs) and piezoelectric types (pMUTs).

Moreover, suitable control circuitry 114 is required for controlling the transducer to emit an acoustic signal having the required properties with respect to e.g. amplitude, pulse shape and timing. However, such control circuitry for ultrasonic transducers is well known to the skilled person and will not be discussed in detail herein.

Each ultrasonic transducer 106 is configured to transmit an acoustic signal ST propagating in the cover structure 102 and to receive a reflected ultrasonic signal S_{R} having been influenced by an object 105, here represented by a finger 105, in contact with the sensing surface 104.

The acoustic interaction signals S_{R} are presently believed to mainly be due to so-called contact scattering at the contact area between the cover structure 102 and the skin of the user (finger 105). The acoustic interaction at the point of contact between the finger 105 and the cover plate 103 may also give rise to refraction, diffraction, dispersion and dissipation of the acoustic transmit signal S_{T}. Accordingly, the interaction signals S_{R} are advantageously analyzed based on the described interaction phenomena to determine properties of the finger 105 based on the received ultrasonic signal. For simplicity, the received ultrasonic interaction signals S_{R} will henceforth be referred to as reflected ultrasonic echo signals S_{R}.

Accordingly, the ultrasonic transducers 106 and associated control circuitry 114 are configured to determine properties of the object based on the received ultrasonic echo signal S_{R}. The plurality of ultrasonic transducers 106 are connected to and controlled by ultrasonic transducer control circuitry 114. The control circuitry 114 for controlling the transducers 106 may be embodied in many different ways. The control circuitry 114 may for example be one central control unit 114 responsible for determining the properties of the acoustic signals S_{T} to be transmitted, and for analyzing the subsequent interaction signals S_{IN}. Moreover, each transducer 106 may additionally comprise control circuitry for performing specified actions based on a received command.

The control unit 114 may include a microprocessor, microcontroller, programmable digital signal processor or another programmable device. The control unit 114 may also, or instead, include an application specific integrated circuit, a programmable gate array or programmable array logic, a programmable logic device, or a digital signal processor. Where the control unit 114 includes a programmable device such as the microprocessor, microcontroller or programmable digital signal processor mentioned above, the processor may further include computer executable code that controls operation of the programmable device. The functionality of the control circuitry 114 may also be integrated in control circuitry used for controlling the display panel or other features of the smartphone 100.

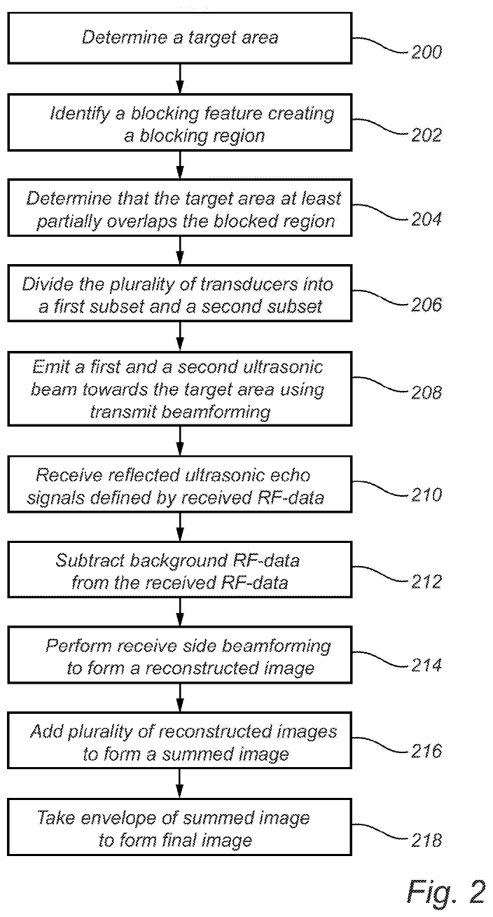

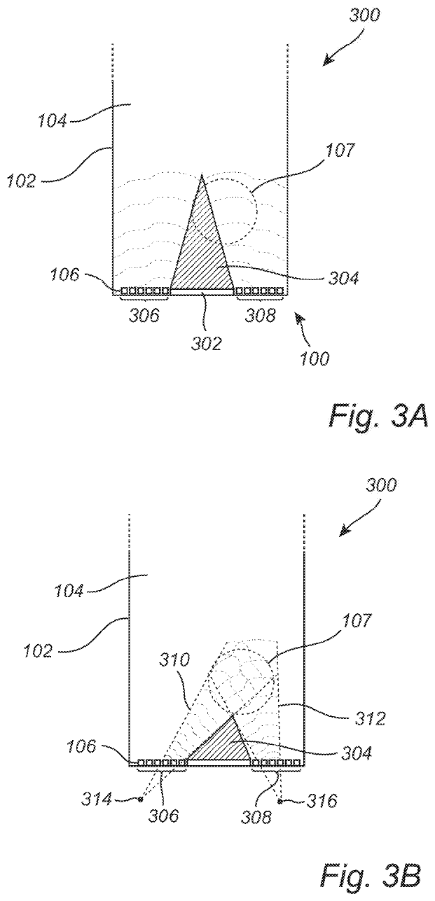

FIG. 2 is a flow chart outlining the general steps of a method for image acquisition in an ultrasonic biometric imaging device 100 according to an embodiment of the invention. The method will be described with reference to the device 100 illustrated in FIGS. 1A-B and to FIGS. 3A-B schematically illustrating a biometric imaging device 100 integrated in a smartphone comprising a blocking feature 302 in the form of a cutout in the cover glass 102 of the display panel.

The first step comprises determining 200 a target area 107 of the touch surface 104. Determining the target area 107 may comprise receiving information describing the target area 107 from a touch sensing arrangement configured to detect a location of an object in contact with the touch surface. The touch sensing arrangement may for example be a capacitive touch panel in a display panel or it may be formed by the ultrasonic transducers.

The following step comprises identifying 202 a blocking feature 302 preventing ultrasonic wave propagation in the touch surface 104 such that the blocking feature 302 creates a blocked region 304 in the touch surface 104 where image acquisition is not possible. The blocked region is thus not a region empty of ultrasonic waves, it is defined as the region where the resolution of the resulting image is insufficient for accurately determine the sought biometric properties, such as ridges and valleys of a fingerprint. Accordingly, the extension of the blocked region 304 may vary depending on the resolution requirement for a given application.

In FIGS. 3A-B, the blocking feature 302 is a preexisting cutout in the display glass which may house a speaker, meaning that no ultrasonic transducers 106 are arranged along the cover glass 102 at the location of the cutout 302. Moreover, the size and shape of the blocking feature can be assumed to be known by the biometric imaging system. Thereby, the step of identifying 202 a blocking feature 302 may comprise acquiring stored information describing properties of the blocking feature 302.

Once the properties of the blocking feature 302 have been determined, it is determined 204 that the target area 107 at least partially overlaps the blocked region 107. If there is no overlap, there is no need for adjusting the emitted ultrasonic beam or beams based on the blocking feature. However, biometric imaging in general may advantageously use the described method comprising transmit and receive beamforming.

If it is determined that there is an overlap between the blocked region 304 and the target area 107 as illustrated in FIG. 3A, the plurality of transducers are divided 206 into a first subset 306 and a second subset 308, the first subset 306 being defined in that ultrasonic waves emitted by the first subset 306 reaches the target area 107 on a first side of the blocking feature 302 and the second subset 308 being defined in that ultrasonic waves emitted by the second subset 308 reaches the target area 107 on a second side of the blocking feature 302, where the second side is here opposite of the first side. In the embodiment illustrated in FIG. 3A, the first subset 306 of transducers is simply selected from the transducers located on the left side of the blocking feature 302 and the second subset 308 of transducers is selected from the transducers located on the right side of the blocking feature 302. The first subset 306 may comprise all of the transducers located to the left of the blocking feature 302, or it may comprise the specific transducers required for providing an ultrasonic beam of the desired shape. In general, the first and second subset of transducers can be considered to be determined by the emission angle of the transducers in relation to the position and size of the blocking feature.

The next step, illustrated in FIG. 3B, comprises controlling 208 the first and second subset 306, 308 of transducers to emit a first and a second ultrasonic beam 310, 312, towards the target area using transmit beamforming, the illustrated ultrasonic beams being defocused ultrasonic beams. The ultrasonic beams may also be unfocused ultrasonic beams.

By means of the transmit beamforming, one or more virtual point sources 314, 316 are formed outside of the cover glass 102 and behind the respective rows of transducers 306, 308. Thereby, defocused ultrasonic beams 310, 312 having a conical shape are formed. Thereby, diffraction of the two ultrasonic beams 310, 312 takes place in a region which is not directly in line of sight form the transducers, effectively reducing the size of the blocked region.

The directionality of the ultrasonic beam is limited by the opening angles of the ultrasonic transducers. The opening angle is inversely proportional to the operating frequency of the transducers such that a higher frequency of the emitted ultrasonic wave leads to a narrower opening angle.

Next, the ultrasonic transducers receive 210 reflected ultrasonic echo signals defined by the received RF-data. As discussed above, the reflected ultrasonic echo signals S_{R} result from interactions with an object in contact with the touch surface at the target area.

In order to more clearly distinguish the echo signal S_{R} in the received RF-data, background RF-data is subtracted 212 from the received RF-data to form what is here referred to as a clean image. The subtraction of the background RF-data from the acquired RF-data can be done either in the raw RF-data or after a receive side beamforming procedure which will be described in further detail below. For subtraction of background RF-data in the RF-data domain, the response of each individual transducer element is stored and a corresponding background measurement for each transducer element is subtracted from the acquired RF-data. It should be noted that all operations are performed in the digital domain, meaning that AD-conversion is performed before subtraction of the background RF-data, and that the background RF-data needs to be available in digital form. The resulting image after subtraction of background RF-data is herein referred to as a clean image.

The background RF-data may be acquired in different ways. The background data may for example be acquired by capturing an image of the entire touch surface either at regular intervals or when it is anticipated that a finger will be placed on the touch surface, for example if prompted by an application in the device. However, capturing an image of the touch surface requires acquiring and storing large amounts of data and if possible, it is desirable to only acquire background data of a subarea of the touch surface corresponding to the target area. This in turn requires prior knowledge of where on the touch surface the finger will be placed.

In a device comprising a capacitive touch screen, it can be possible to use a so-called hover mode of the capacitive touch screen to determine the target are before the actual contact takes place. In the hover mode, the proximity of a finger can be detected, the target area can be anticipated and background RF-data for the anticipated target are can be acquired prior to image acquisition. It would however in principle also be possible to acquire the background noise after the touch has taken place, i.e. when the user removes the finger, even though this may limit the possible implementations of the image acquisition device.

Receive side beamforming to form a reconstructed image from the clean image can be performed 214 either before or after the subtraction of background RF-data described above. The receive side beamforming is performed dynamically by adjusting the delay values of the received echo signals so that they are “focused” at every single imaging pixel. The received signals are focused at any imaging point, which will be repeated until a full image is generated. In general, an example implementation of receive side beamforming referred to as delay-and-sum beamforming can be described by three steps:

1) The delay between each imaging point from the focal point as well as back to each receiving element is estimated.

2) The estimated delay is used in an interpolation step to estimate the RF-data value. The interpolation is used since the delay might be between two samples. For example, a Spline interpolation may be used.

3) The RF amplitudes are summed across all receive channels.

The method further comprises adding 216 a plurality of reconstructed images resulting from a plurality of emitted ultrasonic beams for a given target area to form a summed image. The number of transmit events required for capturing the target area can be estimated based on the relation between the width of the transmitted beam at the target area and the width of the target area. Accordingly, for a focused emitted beam, a larger number of emitted beams is typically required compared to when using an unfocused or defocused beam, assuming that the width of the transmitted beam at the target area is lower than the width of the target area.

The reconstructed images for each transmit event may be either coherently or incoherently added together, i.e. in-phase or out-of-phase depending on if there is a need to reduce the noise in the image (achieved by in-phase addition) or if it is desirable to increase the contrast of the image (can be achieved by out-of-phase addition).

In-phase addition of the reconstructed images can be achieved by converting the received RF-data into in-phase quadrature complex data, IQ-data, thereby making the phase information available. Thereby, reconstructed images represented by IQ data will subsequently be added in-phase (coherently). However, if the reconstructed images should be added out-of-phase (incoherently), IQ data is not needed.

Out-of-phase combining can help to increase the contrast by making sure that the impulse values are always added together without their phase information, i.e. whether they are positive values or negative.

A final image is formed 218 by taking the envelope of the summed image. The final values for every imaging pixel can be either positive or negative due to the nature of the RF-values. However, it is preferred to show the full image based on the brightness of the image. In the RF-values, large values in both positive and negative represent a strong reflectivity and values close to zero represent low reflectivity. Accordingly, envelope detection can be used to convert the original representation into values only in the positive range. However, it should be noted that the step of taking the envelope of the image is optional and that it in some applications is possible to derive sufficient information directly from the summed image.

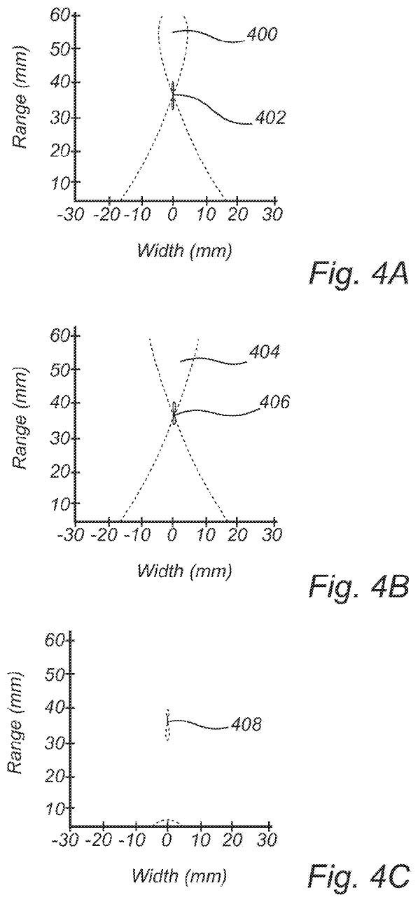

FIG. 4A is a graph showing of the intensity profile 400 of a beamformed shaped ultrasonic transmit beam ST having a focal point 402 approximately at the center of the image, corresponding to a target area.

FIG. 4B is a graph showing of the intensity profile 404 of a beamformed received reflected echo signals SR having a focal point 404 approximately at the center of the image, i.e. at the same location as the focal point 402 of the transmit signal.

FIG. 4C is a graph illustrating the combination of transmit and receive beamforming forming a combined focus point 408 corresponding to a virtual target area. Accordingly, efficient biometric imaging at the target area 107 can be achieved by the combination of transmit and receive beamforming.

FIG. 4A illustrates a focused beam and the same reasoning applies also when emitting a defocused or unfocused beam with the difference that the resulting focus point will be larger. Thereby, since the focus point is larger, fewer transmissions will be required for covering the target area but the resolution will be correspondingly lower. It is thus possible to select whether to use a focused, unfocused or defocused emitted beam based on the requirements of imaging speed vs imaging resolution.

The spatial resolution of the system refers to the ability to resolve points that are very close to each other. In the described system the lateral resolution (x-axis) and the axial resolution (y-axis) is preferably the same. This will make sure that the total resolution is uniform and symmetrical in both directions. The spatial resolution can be represented by a point spread function (PSF) and in the present case the PSF will substantially circular. Biometric image acquisition requires a spatial resolution which is sufficiently high to resolve the features of the biometric object, e.g. to resolve the ridges and valleys of a fingerprint. However, the described method and system may also be used in applications where a much lower resolution is required, e.g. in a touch detection system.

In summary, the described method an and system is useful for improving area coverage of an ultrasonic biometric imaging system in applications where blocking features limits the propagation paths of the emitted ultrasonic signals.

The described method and system can also be useful for expanding the sensing area if there are cracks, scratches or other damage to the surface that influence the imaging properties.

Moreover, the described method and system may advantageously be used in applications which do not comprise a display. In particular, the described method may be used in an application where the touch surface comprises a plurality of openings or other types of blocking features which may not be present in a display screen.

Even though the invention has been described with reference to specific exemplifying embodiments thereof, many different alterations, modifications and the like will become apparent for those skilled in the art. Also, it should be noted that parts of the method and system may be omitted, interchanged or arranged in various ways, the method and system yet being able to perform the functionality of the present invention.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

1. A method for image acquisition in an ultrasonic biometric imaging device, the device comprising a plurality of ultrasonic transducers arranged at a periphery of a touch surface along one side of the touch surface, the method comprising:
determining a target area of a touch surface;
identifying a blocking feature preventing ultrasonic wave propagation in the touch surface such that the blocking feature creates a blocked region in the touch surface where image acquisition is not possible;
determining that the target area at least partially overlaps the blocked region;
dividing the plurality of transducers into a first subset and a second subset, the first subset being defined in that ultrasonic waves emitted by the first subset reaches the target area on a first side of the blocking feature and the second subset being defined in that ultrasonic waves emitted by the second subset reaches the target area on a second side of the blocking feature;
controlling the first and second subset of transducers to emit a first and a second ultrasonic beam towards the target area using transmit beamforming, the ultrasonic beams being defocused or unfocused ultrasonic beams;
by the ultrasonic transducers, receiving reflected ultrasonic echo signals defined by received radio frequency data (RF-data), the reflected ultrasonic echo signals resulting from interactions with an object in contact with the touch surface at the target area;
subtracting background RF-data from the received RF-data to form a clean image;
performing receive side beamforming to form a reconstructed image from the clean image; and
for a plurality of reconstructed images resulting from a plurality of emitted ultrasonic beams for a given target area, adding the plurality of reconstructed images to form a summed image.

2. The method according to claim 1, wherein forming a defocused beam comprises performing transmit beamforming to form a virtual point source located behind the transducers and outside of the touch surface.

3. The method according to claim 1, further comprising emitting a respective first and second directional defocused beam by the first and second subset of transducers such that the blocked region is minimized.

4. The method according to claim 1, further comprising emitting a respective first and second directional defocused beam by the first and second subset of transducers, wherein the first and second directional defocused beam has the same shape.

5. The method according to claim 1, further comprising controlling the ultrasonic transducers to emit a defocused beam or an unfocused beam based on a speed of sound in the touch surface.

6. The method according to claim 1, wherein the touch surface is a surface of a display panel and the blocking feature is an opening in the display panel.

7. The method according to claim 1, wherein identifying a blocking feature comprises retrieving stored information describing properties of the blocking feature.

8. The method according to claim 1, wherein identifying a blocking feature comprises forming an image of at least a portion of the touch surface, detecting a blocking feature in the formed image and determining properties of the blocking feature based on the formed image.

9. The method according to claim 1, wherein emitting a first and a second ultrasonic beam towards the target area using transmit beamforming comprises emitting a first and a second ultrasonic beam having the largest possible angles in relation to the blocking feature.

10. The method according to claim 1, wherein determining the target area comprises receiving information describing the target area from a touch sensing arrangement configured to detect a location of an object in contact with the touch surface.

11. An ultrasonic biometric imaging device comprising:
a cover structure comprising a touch surface;
a plurality of ultrasonic transducers arranged at a periphery of the touch surface, the plurality of ultrasonic transducers being configured to emit a defocused or unfocused ultrasonic beam towards a target area using transmit beamforming and to receive a reflected ultrasonic echo signals defined by received radio frequency data (RF-data), the reflected ultrasonic echo signals resulting from reflections by an object in contact with the touch surface at the target area; and
a biometric imaging control unit configured to:
determine a target area of a touch surface;
identify a blocking feature preventing ultrasonic wave propagation in or at the touch surface such that the blocking feature creates a blocked region in the touch surface where image acquisition is not possible;
determine that the target area at least partially overlaps the blocked region;
divide the plurality of transducers into a first subset and a second subset, the first subset being defined in that ultrasonic waves emitted by the first subset reaches the target are on a first side of the object and the second subset being defined in that ultrasonic waves emitted by the second subset reaches the target area on a second side of the object;
control the first and second subset of transducers to emit a first and a second ultrasonic beam towards the target area using transmit beamforming, the ultrasonic beam being a defocused or unfocused ultrasonic beam;
by the ultrasonic transducers, receive reflected ultrasonic echo signals defined by received RF-data, the reflected ultrasonic echo signals resulting from interactions with an object in contact with the touch surface at the target area;
subtract background RF-data from the received RF-data to form a clean image;
perform receive side beamforming to form a reconstructed image from the clean image; and
for a plurality of reconstructed images resulting from a plurality of emitted ultrasonic beams for a given target area, add the plurality of reconstructed images to form a summed image.

12. The ultrasonic imaging device according to claim 11, wherein the blocking feature preventing ultrasonic wave propagation is a cutout in the cover structure located at the edge of the cover structure, and wherein the first subset of ultrasonic transducers is located at a first side of the cutout and the second subset of ultrasonic transducers is located at a second side of the cutout, opposite the first side.

13. The ultrasonic imaging device according to claim 11, wherein the blocking feature preventing ultrasonic wave propagation is an opening in the cover structure located at the edge of the cover structure

14. The ultrasonic imaging device according to claim 11, wherein the blocking feature preventing ultrasonic wave propagation is a crack in the cover structure located at the edge of the cover structure

15. The ultrasonic imaging device according to claim 11, wherein the cover structure is a display glass.

16. The ultrasonic imaging device according to claim 11, wherein the plurality of transducers are arranged in a single row on a single side of the touch surface.
